(19) United States
(12) Patent Application Publication
(10) Pub. No.: US 2010/0035998 A1
(43) Pub. Date: Feb. 11, 2010

(54) COMBINATION S-NITROSOTHIOL PHARMACEUTICAL PRODUCTS FOR RESTORING NORMAL BREATHING RHYTHMS

(75) Inventor: James C. Mannion, Plainsboro, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Galleon Pharmaceutical, Inc, Horsham, PA (US)

(21) Appl. No.: 11/992,074

(22) PCT Filed: Sep. 20, 2006
(86) PCT No.: PCT/US2006/036846

§ 371 Date: Oct. 15, 2009

Related U.S. Application Data
(60) Provisional application No. 60/718,772, filed on Sep. 20, 2005.

(51) Int. Cl. A61K 31/095; A61P 11/06; A61P 11/16

(52) U.S. Cl. 514/706

(57) ABSTRACT

The present invention is directed to a method of treating a lack of normal breathing control including the treatment of apnea and hypoventilation associated with sleep, obesity, certain medicines and other medical conditions. In an aspect, the invention is directed to treating disordered control of breathing by administering an composition comprising a combination of two or more compounds, at least one of which treats lack of normal breathing. In one aspect, a compound is an S-nitrosylating agent.

### BACKGROUND OF THE INVENTION

Normal control of breathing is a complex process that involves the body's interpretation and response to chemical stimuli such as carbon dioxide, pH and oxygen levels in blood, tissues and the brain. Breathing control is also affected by wakefulness (i.e., whether the patient is awake or sleeping). Within the brain medulla there is a respiratory control center that interprets the various signals that affect respiration and issues commands to the muscles that perform the work of breathing. Key muscle groups are located in the abdomen, diaphragm, pharynx and thorax. Sensors located centrally and peripherally then provide input to the brain's central respiration control areas that enables response to changing oxygen requirements.

Normal respiratory rhythm is maintained primarily by the body's rapid response to changes in carbon dioxide levels (CO₂). Increased CO₂ levels signal the body to increase breathing rate and depth resulting in higher oxygen levels and subsequent lower CO₂ levels. Conversely, low CO₂ levels can result in periods of apnea (no breathing) since the stimulation to breathe is absent. This is what happens when a person hyperventilates.

In addition to the role of the brain, breathing control is the result of feedback from both peripheral and central chemoreceptors—although the exact contribution of each is unknown.

There are a wide variety of diseases that have loss of normal breathing rhythm as a primary or secondary feature of the disease. Examples of a primary loss of breathing rhythm control are: apneas (central, mixed and obstructive where the breathing repeatedly stops for 10 to 60 seconds) and congenital central hypoventilation syndrome. Secondary loss of breathing rhythm may be due to chronic cardio-pulmonary diseases (e.g., heart failure, chronic bronchitis, emphysema, and impending respiratory failure), excessive weight (e.g., obesity-hypoventilation syndrome), certain drugs (e.g., anesthetics, sedatives, anxiolytics, hypnotics, alcohol, narcotic analgesics) and/or factors that affect the neurological system (e.g., stroke, tumor, trauma, radiation damage, ALS). In chronic obstructive pulmonary diseases where the body is exposed to chronically low levels of oxygen the body adapts to the lower pH by a kidney mediated retention of bicarbonate which has the effect of partially neutralizing the CO₂/pH respiratory stimulation. Thus, the patient must rely on the less sensitive oxygen-based system.

In particular, loss of normal breathing rhythm during sleep is a common condition. Sleep apnea is characterized by frequent periods of no or partial breathing. Key factors that contribute to these apneas include: decrease in CO₂ receptor sensitivity, decrease in hypoxic ventilatory response sensitivity (e.g., decreased response to low oxygen levels) and loss of “wakefulness”. Taken together normal breathing rhythm is disturbed resulting in hypoxia (and the associated oxidative stress) and eventually severe cardiovascular consequences (high blood pressure, stroke, heart attack). Snoring has some features in combination with sleep apnea. The upper airway muscles lose their tone resulting in the sounds associated with snoring but also inefficient airflow which may result in hypoxia.

The definitive treatment for many breathing control disorders is either mechanical ventilation or positive airway pressure devices (e.g., continuous positive airway pressure device (CPAP device), bi-level positive airway pressure device (BiPAP device)). Several pharmacologic agents have been proposed as interventions to control respiration in sleep-related breathing disorders. De Backer provided a review and described Progestin, Almitrine and Acetazolmide (DeBacker W A. 1995 Eur. Respir. J. 8:1372-1383). Hudgel and Thanakitcharu also provided a review of pharmacologic treatment of sleep disordered breathing and described medroxyprogesterone, thyroid replacement, acetazolamide, theophylline, tricyclic antidepressants, serotonin reuptake inhibitors and clonidine in addition to other agents (Hudgel D W and Thanakitcharu S. 1998 Am J Respir Crit. Care Med 158:691-699). In 2005 Qureshi and Lee-Chiong provided a review of various medical options to treat obstructive sleep apnea, including a wide variety of pharmacological treatments. A few of the agents included benzodiazepines, narcotics, acetazolamide, antidepressants and agents that affect serotonin as either agonists, re-uptake inhibitors or antagonists (Qureshi A and Lee-Chiong, JR, T L Sem. Resp Crit. Care Med 2005; 26: 96-108.

In particular, DeBacker noted that low doses of the carbonic anhydrase inhibitor acetazolamide seemed to exert a beneficial effect not related to its traditional action of reducing pH as a mechanism of respiratory stimulation. In a small, uncontrolled clinical study in central apnea patients low doses of acetazolamide were found to decrease apnea episodes from 25.5 pre-treatment to 6.8 after one month of treatment (73%). Smaller reductions (about 25%) were seen in patients that had predominantly obstructive sleep apnea.

More recently, Carley and Radulovacki described the use of a serotonin agonist/antagonist combination to increase motor tone in the portion of the throat that collapses in obstructive sleep apnea (Carley and Radulovacki, 1999, Am. J. Respir. Crit. Care Med. 160:1824-1829). This concept is currently in commercial development by a partnership comprised of Organon and Cypress Bioscience and a separate group, BTG, plc (see, for example, U.S. Patent Application Publication Numbers 20060039866, 20060039867, 20060122127).

Gaston and Gozal proposed a fundamentally different approach by demonstrating that the S-nitrosothiol signaling pathway can be used to exert control over respiration by increasing minute ventilation (International Patent Application Publication No. WO 03/015605, the entirety of which is incorporated by reference herein). They demonstrated, for the first time, that the centrally-mediated hypoxic ventilatory response system is under the control of certain S-nitrosothiol compounds. Gaston and Gozal demonstrate a group of compounds that can induce the body's typical response to low oxygen levels triggering, among other reactions, increases in the rate and depth of breathing.

The ability of a mammal to breathe, and to modify breathing according to the amount of oxygen available and demands of the body, is essential for survival. There are a variety of conditions that are characterized by loss of respiratory rhythm due to either a primary or secondary cause. Estimates for afflicted individuals for several of the most frequent conditions in the United States include, sleep apneas: 15-20 million; obesity-hypoventilation syndrome: 5-10 million; chronic heart disease: 5 million; chronic obstructive pulmonary disease (COPD)/chronic bronchitis: 10 million; drug-induced hypoventilation: 2-5 million; and mechanical ventilation weaning: 0.5 million.

The control of breathing is a complex process. It involves respiratory drive, and also the diameter of the tubes through which air flow occurs. For example, assume an animal is breathing through a straw. If the straw is dry and the walls are rigid, air will flow smoothly both during inspiration (negative pressure) and exhalation (positive pressure). However, if the straw becomes wet, during inhalation, the walls collapse and the animal will not be able to inhale any air. This “wet straw” example is partially descriptive of what happens in patients afflicted with sleep apnea. When a patient with sleep apnea goes to sleep, respiratory drive decreases and the muscle tone in the airway decreases and the airway collapses during inspiration, causing an obstruction to normal breathing. Current treatment for sleep apnea is primarily the use of positive airway pressure (PAP) devices. Compliance with these devices is usually very poor. Pharmaceutical products that could be used either alone or as an adjuvant to positive airway pressure devices thereby lowering the pressure requited to maintain airway patency would be an important advance to either improve compliance with these PAP devices or provide an alternative means of treatment.

Accordingly, a combination pharmaceutical product that could restore all or part of the body's normal breathing control system in response to changes in CO₂ and/or oxygen would be of benefit in decreasing the incidence and severity of breathing control disturbances. There is currently an unmet need for such a product that can be administered to a patient with minimal side effects. The present invention addresses and meets this need.

### BRIEF SUMMARY OF THE INVENTION

The invention includes a therapeutic composition for stabilizing breathing rhythm, comprising a first composition comprising a S-nitrosothiol first compound and a second composition comprising a second compound that is not an S-nitrosothiol compound, wherein the second compound has the activity of stabilizing breathing rhythm.

In an aspect, the second compound is selected from the group consisting of a carbonic anhydrase inhibitor, a serotonin agonist, a serotonin antagonist, an NADPH oxidase inhibitor, a leukotriene antagonist, a COX-2 inhibitor and theophylline. In one embodiment, a carbonic anhydrase inhibitor is selected from the group consisting of acetazolamide and topiramate. In another embodiment, the second compound is a tetracyclic antidepressant selected from the group consisting of mirtazipine and setiptiline.

In another embodiment, a serotonin agonist is selected from the group consisting of mirtazapene, buspirone and a serotonin re-uptake inhibitor. In an embodiment, a serotonin antagonist is ondansetron.

In another embodiment, the invention includes an NADPH oxidase inhibitor selected from the group consisting of apocynin, 4-hydroxy-3′-methoxyacetophenon, N-vanillylnonanamide, and staurosporine.

The invention includes a method of stabilizing the breathing rhythm of a mammal, comprising administering to a mammal the therapeutic composition comprising a first composition comprising a S-nitrosothiol first compound and a second composition comprising a second compound that is not an S-nitrosothiol compound, wherein the second compound has the activity of stabilizing breathing rhythm.

The invention includes a therapeutic composition further comprising a third compound, wherein the third compound is an S-nitrosothiol compound. The invention also includes a therapeutic composition further comprising a third compound, wherein the third compound is not an S-nitrosothiol compound.

The invention includes a pharmaceutical composition comprising the composition as set forth herein and a pharmaceutically-acceptable carrier.

The invention includes a method of stabilizing the breathing rhythm of a mammal, comprising administering to a mammal a therapeutic composition as described herein.

The invention also includes a method of stabilizing the breathing rhythm of a mammal, said method comprising administering to a mammal the therapeutic composition of claim 1, said method further comprising treating said mammal with a ventilation assist device. In an embodiment, the ventilation assist device is selected from the group consisting of a CPAP device and a BiPAP device.

In a method of the invention, the route of administration is selected from the group consisting of parenteral, oral and buccal. In an embodiment, a parenteral route of administration is selected from the group consisting of transdermal, intravenous, intramuscular, and intradermal. In another embodiment, a composition is administered by at least two routes of administration.

The invention includes a method of increasing minute ventilation (V_{E}) at the level of the brainstem respiratory control centers in the nucleus tractus solitarius of an individual, comprising the step of administering to an individual a therapeutic composition comprising a first composition comprising a S-nitrosothiol first compound; and a second composition comprising a second compound that is not an S-nitrosothiol compound, wherein the second compound has the activity of increasing minute ventilation (V_{E}) at the level of the brainstem respiratory control centers in the nucleus tractus solitarius.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the complex and interconnected nature of mammalian breathing control.

FIG. 2, comprising FIGS. 2A-2C, illustrates the factors affecting breathing control. FIG. 2A illustrates factors affecting normal breathing control. Normally respiratory drive can operate across a range of conditions, and carbon dioxide and oxygen levels are the main drivers and act in an interrelated manner. FIG. 2B illustrates factors affecting disordered breathing control. A wide range of factors act individually or in combination to decrease respiratory drive resulting in breathing stoppages or inefficient breathing. The eventual outcome is hypoxia which leads to cardiovascular, neurological and/or metabolic consequences. FIG. 2C illustrates factors to be considered for effective pharmacotherapy of disordered breathing control. Drugs are useful to help restore respiratory drive through the defined pathways or by improving airflow in the upper airway. The eventual outcome, in one embodiment of the invention, is to decrease disordered breathing (e.g., apnea, hypopnea, hypoventilation), hypoxia and the associated consequences.

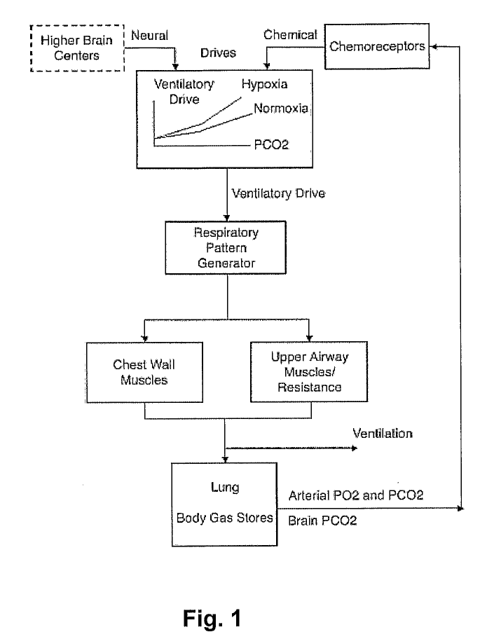

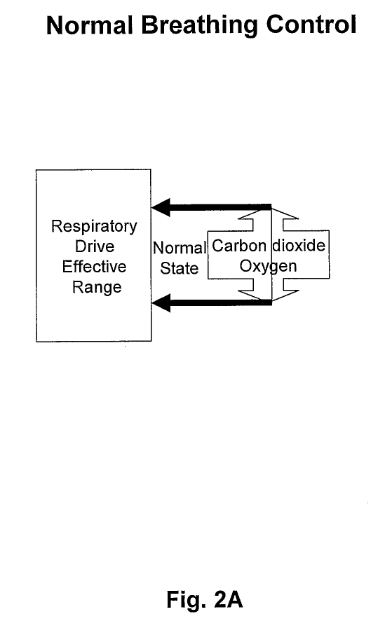

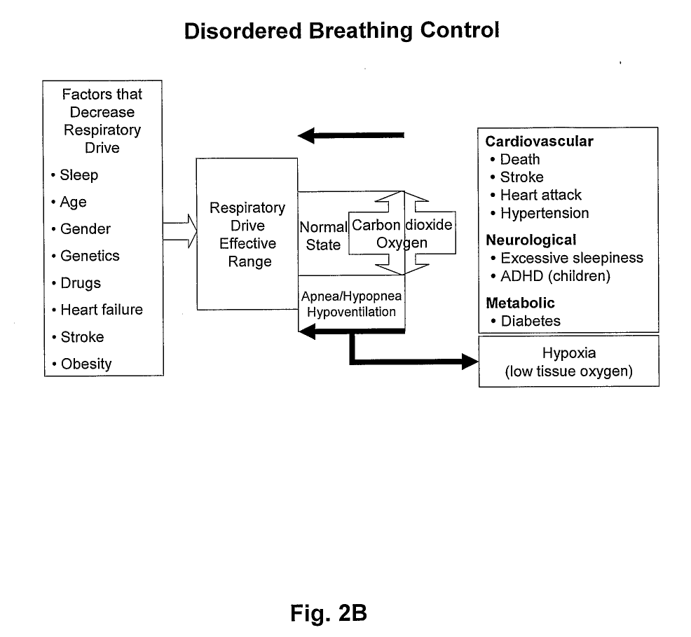

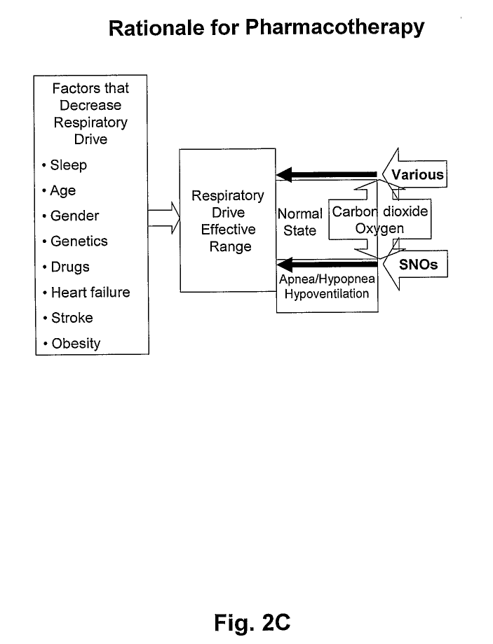

### DETAILED DESCRIPTION

The present invention relates to a combination, or “multi-drug,” approach to the treatment of sleep apnea by combining hypoxic ventilatory response control, by way of administration of S-nitrosothiols, with other drugs that provide a complimentary activity.

The invention provides that a composition comprising a combination of two or more compounds may provide enhanced effectiveness in the treatment of disorders of breathing control by acting upon on two or more physiological pathways, wherein one of the pathways is affected by S-nitrosothiol treatment for restoration of respiratory rhythm. In another aspect of the invention, a composition comprising a combination of two or more compounds may provide enhanced effectiveness in the treatment of disorders of breathing control by acting upon the same physiological pathway.

Poor or inefficient respiratory drive results in hypoventilation, which further results in hypoxia. A primary initial clinical manifestation of hypoxia is drowsiness or excessive daytime sleepiness. Accordingly, drugs that cause decreased respiratory drive and the resulting hypoxia are sometimes limited in their usefulness due to the fear of a life-threatening respiratory depression and/or the excessive daytime sleepiness that negatively impacts quality of life. Another outcome of hypoxia from respiratory drive deficiency is oxidative stress which has been linked to longer term cardiovascular and/or metabolic outcomes. Combination products that combine a compound to restore respiratory rhythm with an agent that helps reduce oxidative stress can provide an important dual mode of action to alleviate short and long-term consequences of hypoxia.

As set forth herein, combination compositions that comprise an S-nitrosothiol compound to counteract the respiratory depressant effect of drugs that decrease respiratory drive can provide a benefit to patients by helping to maintain normal oxygen levels in the blood and tissues. By way of a non-limiting example, narcotic analgesics (e.g., morphine, fentanyl, oxycodone, buprenorphine) are administered to cancer patients to alleviate pain. The dose is often limited by a fear of respiratory depression. In addition, even a partial respiratory depression from these drugs causes hypoxia and a resulting excessive daytime sleepiness that can be debilitating and severely decrease quality of life. General anesthetics can exert a similar depressant effect on respiration and delay a patient's transfer from the operating room to a surgical recovery area. A combination composition comprising an S-nitrosothiol compound is therefore useful to counteract the lingering effects of the anesthetic, and for restoring adequate respiratory drive to enable the patient to breathe on their own.

By way of another non-limiting example, excessive weight can decrease respiratory drive which results in hypoventilation and hypoxia. This condition is called obesity-hypoventilation syndrome. Excessive weight is also a risk factor in sleep related breathing disorders. A combination composition comprising an S-nitrosothiol compound is therefore useful to counteract the respiratory depressant effects of obesity.

Combination compositions of the invention are also useful for increasing the muscle tone of the upper airway, improving ventilatory/perfusion match and increasing erythropoietin production, among other things, as set forth in detail herein.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “apnea” means the absence of normal breathing resulting in intermittent stoppages of breathing.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

“Cheyne-Stokes respiration” refers to a specific pattern of breathing characterized by a crescendo pattern of breathing that results in apneas and/or hypopneas. A hallmark of this condition is that breathing becomes out of phase with blood oxygen levels.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Hypopnea” is similar in many respects to apnea; however, breathing does not fully stop but is partially stopped (i.e., less than 100% of normal breathing, but more than 0% of normal breathing). Hypopnea is also referred to herein as “partial apnea” and can be subdivided into obstructive, central or mixed types.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “modulate” is meant to refer to any change in biological state, i.e. increasing, decreasing, and the like.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, a “therapeutically effective amount” is the amount of a therapeutic composition sufficient to provide a beneficial effect to a mammal to which the composition is administered.

“S-Nitrosothiol pathway,” as the term is used herein, refers to the signaling pathway and the signaling mechanisms that occur as the information pertaining to blood levels of oxygen is transmitted to the brain through S-nitrosothiol signaling.

### DESCRIPTION

### Compositions of the Invention and Uses Thereof

The present invention includes compositions and methods for treating disordered control of breathing. In one embodiment, the invention provides methods and compositions for treating sleep apnea.

During sleep, breathing changes with the stage or depth of sleep. Some individuals stop breathing for brief intervals. When such episodes of apnea become more frequent and last longer, they can cause the body's oxygen level to decrease, which can disrupt sleep. The patient may not fully awaken, but is aroused from the deep restful stages of sleep, and thus feels tired the next day.

There are two main types of sleep apnea which may occur together. The most common is obstructive sleep apnea, during which, breathing is blocked by a temporary obstruction of the main airway, usually in the back of the throat. This often occurs because the tongue and throat muscles relax, causing the main airway to close. The muscles of the chest and diaphragm continue to make breathing efforts, but the obstruction prevents any airflow. After a short interval lasting seconds to minutes, the oxygen level drops, causing breathing efforts to become more vigorous, which eventually opens the obstruction and allows airflow to resume. This often occurs with a loud snort and jerking of the body, causing the patient to arouse from deep sleep. After a few breaths, the oxygen level returns to normal, the patient falls back to sleep, the muscles of the main airway relax and the obstruction occurs again. This cycle is then repeated over and over during certain stages of sleep. Most people with obstructive sleep apnea snore, suggesting that their main airway is already partly obstructed during sleep, but not all people who snore have obstructive sleep apnea.

A less common form of sleep apnea is central sleep apnea, so named because the central control of breathing is abnormal. This control center lies in the brain, and its function can be disrupted by a variety of factors. There is no obstruction to airflow. The patient with sleep apnea stops breathing because the brain suddenly fails to signal the muscles of the chest and diaphragm to keep breathing. These patients do not resume breathing with a snort and body jerk, but merely start and stop breathing at various intervals. Although the mechanism is different than obstructive sleep apnea, sleep is still disturbed by the periodic decreases in oxygen, and the patients suffer from the same daytime symptoms.

Some patients may suffer from a combination of the two causes of apnea, a disorder which is called mixed-sleep apnea, also termed “complex sleep apnea.”

Sleep apnea should be suspected in individuals who are noted to have excessive daytime sleepiness and other symptoms described above, especially if they are known to snore and have a restless sleep. Commonly, these patients have exhibited loud snoring for many years, more often are male, and note that the daytime sleepiness has become a progressive problem over many months. Less commonly, they may be bothered by bedwetting or impotence. The sleep problems are often aggravated by alcohol or sedative medications. They are also more readily noticed by the patient's family and friends, especially the bed partner.

The compounds and methods of the present invention should be understood to be applicable to any other respiratory control that is associated with an S-nitrosothiol signaling pathway. That is, the present invention provides that a composition comprising a combination of two or more compounds may provide enhanced effectiveness in the treatment of disorders of breathing control by acting upon on two or more physiological pathways, wherein one of the pathways is affected by S-nitrosothiol treatment for restoration of respiratory rhythm.

In another aspect of the invention, a composition comprising a combination of two or more compounds may provide enhanced effectiveness in the treatment of disorders of breathing control by acting upon the same physiological pathway. In one aspect of the invention, a composition is used to treat sleep apnea.

According to the present invention, the second compound used in conjunction with an S-nitrosothiol compound, can be selected for a specific property or activity, as described in detail herein. In one aspect of the invention, the third, fourth, or additional compound can similarly be a non-5-nitrosothiol compound, selected for a specific property or activity, as described in detail herein. The following are non-limiting examples of such compounds, but should not be considered in any way to be the only such compounds useful in the present invention. The skilled artisan, when armed with the present disclosure, will understand how to identify a second (or third, fourth, fifth, etc . . . ) compound useful in combination with an S-nitrosothiol compound according to the present invention.

Table 1. Examples of Compounds Useful in Combination with an S-Nitrosothiol Compound According to the Present Invention
a. A compound with the activity of stabilizing breathing rhythm
   i. Carbonic anhydrase inhibitor (e.g., acetazolamide, topiramate)
   ii. Respiratory stimulation (e.g., caffeine, theophylline, doxapram)
   iii. Narcotic antagonists (e.g., naloxone)
   iv. Hormones (e.g., medroxyprogesterone)
b. A compound with the activity of increasing the patency of the upperairway by activity on serotonin, dopamine, norepinephrine or GABA
   i. Serotonin agents (e.g., 5HT1A agonist buspirone, serotonin re-uptake inhibitors, 5HT3 receptor antagonists such as ondansetron)
   ii. Dopamine and/or norepinephrine agents (e.g., ropinerole, milnacipran)
   iii. Tetracyclic antidepressants (e.g., mirtazipine, setiptiline)
c. A compound with the activity of promoting wakefulness
   i. Modafinil, r-modafinil, amphetamine
d. A compound with the activity of decreasing seizures
   i. Zonisamide
e. A compound with the activity of increasing the patency of the upper airway by decreasing inflammation
   i. Antihistamines (e.g., cetirizine, azelastine, desloratidine, fexofenadine)
   ii. Leukotriene antagonists (e.g., montelukast)
   iii. 5-lipoxygenase inhibitors (e.g., zileuton)
   iv. Steroids (e.g., fluticasone)
   v. COX-2 inhibitors
f. A compound with the activity of decreasing respiratory drive as a side effect to its primary therapeutic effect
   i. Opoid analgesics (e.g., morphine, meperidine, fentanyl, oxycodone, buprenorphine)
   ii. Sedative hypnotics (e.g., lorazepam, zolpidem, zaleplon)
   iii. General anesthetics (e.g., halothane, enflurane, thiopental)
   iv. Ethyl alcohol
g. A compound with the activity of improving lung function in diseases such as asthma and/or chronic obstructive pulmonary disease,
   i. Steroids (e.g., budesonide, fluticasone, salmeterol/fluticasone combinations)
   ii. Bronchodilators (e.g., salbutamol, salmeterol)
   iii. Anticholinergic (e.g., tiotropium, ipatropium)
h. A device use to assist breathing through mechanical ventilation or positive airway pressure.
   i. Mechanical ventilators
   ii. CPAP
   iii. BiPAP

Combination drugs in the pharmaceutical industry are reasonably common, and the preparation and use of such drugs will be understood by the skilled artisan. For example, Advair® is a combination of a steroid compound and a bronchodilating compound, and is used for treatment of asthma.

Combinations comprising two or more compounds according to the present invention include, but are not limited to, S-nitrosothiol compounds+acetazolamide (and other carbonic anhydrase inhibitors including topiramate), S-nitrosothiol compounds+serotonin agonist agents (e.g., 5HT1A agonist buspirone; serotonin re-uptake inhibitors), S-nitrosothiol compounds+serotonin antagonist agents (e.g., 5HT3 receptor antagonists, such as ondansetron), S-nitrosothiol compounds+tetracyclic antidepressants (e.g., mirtazipine, setiptiline) S-nitrosothiol compounds+modafinil, S-nitrosothiol compounds+r-modafinil, S-nitrosothiol compounds+compounds that effect the neuronal uptake of norepinephrine and/or dopamine (e.g., ropinerole, milnacipran), S-nitrosothiol compounds+zonisamide, S-nitrosothiol compounds+agents that stimulate brain activity and/or are opoid antagonists (e.g., doxapram, naloxone, caffeine), S-nitrosothiol compounds+narcotic analgesics that cause respiratory depression (e.g., morphine, meperidine, fentanyl, oxycodone, buprenorphine), S-nirosothiol compounds+general anesthesics that cause respiratory depression (halothane, enflurane, thiopental), S-nitrosothiol compounds+theophylline S-nitrosothiol compounds+steroid and/or bronchodilator agents commonly used to treat asthma or chronic obstructive pulmonary disease (e.g., budesonide, fluticasone, salbutamol, formoterol, salmeterol/fluticasone combinations, tiotropium, ipatropium), S-nitrosothiols+antihistamines (e.g., cetirizine, azelastine, desloratidine, fexofenadine), S-nitrosothiol compounds+sedative/hypnotics (e.g., lorazepam, zolpidem, zaleplon), and S-nitrosothiol compounds in combination with positive airway pressure breathing devices (including CPAP and BiPAP). Other compounds useful in combination with S-nitrosothiol compounds, as set forth herein, are described in U.S. Patent Application Publication No. 20060039866, which is incorporated by reference herein in its entirety.

In one embodiment of the invention, a combination of two or more compounds, wherein at least one compound acts through the S-nitrosothiol pathway would provide an additive or synergistic effect to restore normal breathing rhythm. In another embodiment of the invention, a combination of two or more compounds, wherein at least one compound acts through the S-nitrosothiol pathway, provides an effect to counteract the respiratory depressant effect of another drug that may or may not be administered at the same time.

S-Nitrosothiol compounds, or “SNOs,” have been described to have various clinical benefits. These include, but are not limited to, increase in respiratory drive, increase in muscle tone in the upper airway, improvement of oxygen exchange in the lungs (“ventilatory perfusion matching”), and increased production of erythropoietin (EPO), a natural hormone that increases red blood cell production. Increased EPO production may be especially useful in patients that have breathing problems (with the accompanying hypoxia) and anemia. Such conditions result in a “double negative effect” of low oxygen levels and a low count of cells that carry the oxygen (E.g., apnea of prematurity, kidney dialysis patients).

In one aspect of the invention, a compound of the invention is useful in the form in which the compound is administered. That is, the chemical structure and formula of the compound that is administered to the patient is the compound that is active according to a method of the invention. In another aspect, a compound of the invention is active in a form other than that structure or formula which is administered to a patient. In this aspect of the invention, a compound must first be altered, added to, broken down, metabolized or otherwise modified from the form in which the compound is administered to the patient. By way of a non-limiting example, N-acetylcysteine (NAC) is one such compound. NAC is administered to a patient as a pro-drug, which is metabolized by the body to S-nitrosothiol-N-acetylcysteine (SNOAC). SNOAC, the metabolized compound, is subsequently active in ordering breathing of a patient, for example. See, for example, International Patent Application Publication No. WO 03/015605, the entirety of which is incorporated by reference herein.

In another aspect of the invention, the S-nitrosothiol compound is an analog, derivative or modification of a known S-nitrosothiol compound. By way of several non-limiting examples, an S-nitrosothiol compound encompassed by the present invention includes an analog of N-acetylcysteine, a derivative of N-acetylcysteine, a modification of N-acetylcysteine, and a metabolite of N-acetylcysteine.

It will be understood by the skilled artisan, when armed with the disclosure set forth herein, that analogs and derivatives of S-nitrosothiol compounds can be prepared and used according to the invention set forth herein. The skilled artisan will understand how to identify which portion or portions of an S-nitrosothiol compound to modify, and further, how to make such modifications, in accordance with the present invention. Additionally, based on the detailed description set forth herein, the skilled artisan will know how to assay such compounds to identify analogs or derivatives that have the activity of a compound of the invention, namely, the ability to control breathing in accordance with the present invention, when used in combination with one or more additional compounds.

By way of a non-limiting example, a combination according to the invention comprises acetazolamide combined with N-acetylcysteine. In an embodiment, a combination according to the invention comprises a low dose of acetazolamide (e.g., 250 mg/day or less) combined with N-acetylcysteine. While not wishing to be bound by any particular theory, a combination of acetazolamide and N-acetylcysteine may work in a complimentary or synergistic manner to restore normal respiratory rhythm. Acetazolamide can work to restore the body's sensitivity to CO₂ and N-acetylcysteine can work to restore the sensitivity of the breathing centers to low oxygen levels.

Acetazolamide has been used for many years as a mild diuretic (i.e., to increase urine output or to help treat mountain sickness). Acetazolamide is also believed to work through the carbon dioxide based respiratory drive pathway. It is proposed to work by lowering the pH of the blood, but this may not be the only way it affects respiratory drive. Decreases in respiratory drive may be caused by poor function of the carbon dioxide component, the oxygen component, or both components together. These components are, in fact, interrelated and causing an effect on one may affect the other and the overall respiratory drive.

Therefore, in one embodiment of the invention, in cases such as sleep apnea where both CO₂ and O₂ drive is diminished, a combination composition is used to provide a clinical benefit and/or treatment of the patient. That is, in one embodiment, the invention provides a method of treating sleep apnea.

Traditional thinking was previously that the doses of acetazolamide needed for treatment are too toxic for long-term use in a large number of patients. However, lower doses of acetazolamide may be sufficient to produce the desired effects on respiratory drive, particularly in combination with one or more other components according to the present invention. Other compounds that may be more effective at lower doses, due to the prevalence of side effects when used at higher doses, include, but are not limited to theophylline.

A combination composition according to the invention is useful to treat any condition characterized by lack of normal breathing control. By way of a non-limiting example, such conditions include, sleep apnea (central, mixed and obstructive including but not limited to co-existing conditions of heart failure, kidney disease and stroke), sleep-disordered breathing (especially with snoring and arousals), chronic bronchitis, COPD, asthma, allergy and neurological diseases (e.g., stroke, amyotrophic lateral sclerosis (ALS)). Other conditions that can be treated with the methods and compositions of the present invention include, but should not be limited to, snoring, obesity-hypoventilation syndrome, apnea of prematurity, respiratory depression due to drugs (e.g., narcotic analgesics, sedatives, alcohol, sleeping pills, anesthetics), central congenital hypoventilation syndrome, hypoventilation due to stroke, trauma, surgery and/or radiation, and acclimatization to high altitude.

A combination composition according to the invention is also useful to assist in the treatment of any condition that is treatable using a positive airway pressure (PAP) device, as described elsewhere herein.

By way of a non-limiting example, the present invention may also be used to treat and/or alleviate symptoms of, or to facilitate, acclimatization to high altitude. Genetic diversity plays a role in how people respond to low oxygen levels. Some respond quickly by increasing the rate and depth of breathing (the hypoxic ventilatory response) while some others are slower. There are some cases where the ability to adapt quickly is important. For example, soldiers quickly inserted into a battle situation at high altitude (e.g., 12,000 feet in Afghanistan) need to operate at peak performance. A slow response to hypoxia will result in excessive tiredness and poor work performance. For soldiers this may be life-threatening. For the extreme altitude mentioned the case is fairly clear-cut. There also may be application at lesser altitudes such as the transition from New York to Denver (5,000 ft) or the jet lag from a long airplane flight (cabin pressure of 6,000 feet).

Serotonin agonist or re-uptake inhibitor compounds (e.g., Mirtazapine) have been demonstrated in animals to help restore the tone of the upper airway to prevent collapse. In an aspect of the invention, an SNO/serotonin agonist combination composition is used, whereby the SNO is used to improve respiratory drive, and the serotonin agonist improves the upper airway tone to help air flow and help prevent obstruction.

In another embodiment, the invention includes a combination of a SNO with an agent intended to reduce oxidative stress. When the body stops breathing and oxygen levels drop, there are a series of reactions leading to oxidative stress that is believed to be directly causative of the cardiovascular complications associated with sleep apnea and other conditions. The cardiovascular complications are the main cause of death.

In an aspect of the invention, a combination composition comprises N-acetylcysteine, which is used to reduce oxidative stress through a metabolic pathway unrelated to SNO production. That is, the invention also includes methods and combination compositions in which N-acetylcysteine or another SNO-containing compound reduces oxidative stress in combination with another drug, either a second SNO, or a non-SNO compound such as, but not limited to, acetazolamide, wherein the second compound acts to increase respiratory drive. Other combinations useful in the methods and compositions of the invention will be understood by the skilled artisan, when armed with the disclosure set forth in the present disclosure.

In another aspect, the invention includes a combination composition comprising an SNO compound and a compound that treats and/or prevents oxidative stress in a mammal. In one embodiment, the invention includes a method of treating a patient lacking normal breathing by administering a compound of the invention.

Frequent hypoxia/reoxygenation events, which replicate oxygenation patterns in sleep apnea, induce in one embodiment NADPH oxidase and proinflammatory gene expression in select brain regions, including in another embodiment, in wake-active neurons. In one embodiment, lack of a functional NADPH oxidase and pharmacological inhibition of NADPH oxidase is determined to confer resistance to intermittent hypoxia-induced neurobehavioral, redox and pro-inflammatory changes, thereby emphasizing a potential target to prevent oxidative morbidities in persons with obstructive sleep apnea (OSA).

U.S. Patent Application Publication No. 20060154856 (which is incorporated herein by reference in its entirety) identifies NADPH oxidase as an important source of intermittent hypoxia-induced injury in the brain. In another embodiment, NADPH oxidase activation in persons with OSA contributes to the cardiovascular morbidities associated with this disease. The NADPH oxidase pathway is therefore a valuable pharmacotherapeutic target for both neurobehavioral and cardiovascular morbidities of the prevalent disorder, sleep apnea. According to one aspect of the present invention, the invention provides a method for treating a cardiovascular morbidity, a neurobehavioral morbidity or a combination thereof, resulting from sleep apnea hypopnea syndrome in a subject, comprising administering to said subject a therapeutically effective amount of a composition comprising an NADPH oxidase inhibitor, and at least one other compound. In one embodiment, the at least one other compound is an inhibitor of the S-nitrosothiol signaling pathway. NADPH oxidase inhibitors include, but are not limited to, apocynin, or 4-hydroxy-3′-methoxyacetophenon, N-vanillylnonanamide, and staurosporine.

In another embodiment, the invention includes a combination of a SNO with an agent intended to reduce inflammation. Examples include a leukotriene receptor antagonist (or 5-lipoxygenase inhibitor), antihistamine or anti-inflammatory agent (e.g., COX-2 inhibitor or steroid). In one aspect, the invention includes a method of using such a combination composition to treat a patient lacking normal breathing.

Patients with sleep disordered breathing have turbulent airflow that causes inflammation and reduces their ability to efficiently get air. As discussed elsewhere herein, SNO compounds increase respiratory drive and may increase the diameter of the upper airway passages. Therefore, according to the invention, a combination composition comprising a SNO plus an anti-inflammatory compound is useful to provide a complimentary therapeutic benefit (Goldbart et al, Am. J. Respir. Crit. Care. Med. 2005; 172: 364-370).

By way of a non-limiting example, leukotriene antagonist therapy, using compositions and methods of the present invention, will decrease inflammation that results from turbulent airflow, ordering the breathing of a patient suffering from lack of normal breathing. This is because disturbed airflow causes inflammation which further restricts airflow, since the inflammation decreases the size of the airway passages. According to the present invention, combination composition products that include an anti-inflammatory agent are useful to provide an additional benefit for both adult and pediatric patients with various forms of sleep disordered breathing.

In one aspect of the invention, a combination product of a SNO prodrug (for example, N-acetylcysteine) or a SNO in combination with a leukotriene antagonist (or a 5-lipoxygenaseoxidase inhibitor) are useful to treat disordered control of breathing, while at the same time, minimizing the inflammation associated with such breathing disorders.

In another aspect of the invention, the invention includes a combination composition comprising three or more compounds for the treatment of a disease or disorder involving a lack of normal breathing control. The invention also includes methods for treating a mammal, wherein the method uses a combination composition comprising three or more compounds for the treatment of a disease or disorder involving a lack of normal breathing control. A composition according to the invention may comprise one or more SNO compounds. In another embodiment, a composition according to the invention may comprise three or more non-SNO compounds. Compounds useful in a combination composition of the invention are described in detail elsewhere herein.

In another aspect of the invention, a method of treating a patient lacking normal breathing comprises administering a compound of the invention, as described herein, and additionally treating the patient using a device for treatment of a lack of normal breathing. As described in detail elsewhere herein, such devices include, but are not limited to, CPAP and BiPAP devices.

### Pharmaceutical Compositions

The invention also encompasses the use of pharmaceutical compositions of an appropriate protein or peptide and/or isolated nucleic acid to practice the methods of the invention. The compositions and combinations of compounds set forth herein can be used alone or in combination with additional compounds to produce additive, complementary or synergistic effects in the treatment of disordered breathing, and in the treatment of sleep-related breathing disorders.

In an embodiment, the pharmaceutical compositions useful for practicing the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer compounds according to the methods of the invention.

Compounds which are identified using any of the methods described herein, and combinations of such compounds, may be formulated and administered to a mammal for treatment of disordered control of breathing.

Such a pharmaceutical composition may consist of the active ingredient alone, in a form suitable for administration to a subject, or the pharmaceutical composition may comprise at least one active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Controlled- or sustained-release formulations of a pharmaceutical composition of the invention may be made using conventional technology. Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

The source of active compound to be formulated will generally depend upon the particular form of the compound. Small organic molecules and peptidyl or oligo fragments can be chemically synthesized and provided in a pure form suitable for pharmaceutical usage. Products of natural extracts can be purified according to techniques known in the art. Recombinant sources of compounds are also available to those of ordinary skill in the art.

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

Liquid derivatives and natural extracts made directly from biological sources may be employed in the compositions of this invention in a concentration (w/v) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound for use in the invention in the aqueous gel formulation. Preferred antioxidants for some compounds are BHT, BHA, alphatocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Controlled-release preparations may also be used and the methods for the use of such preparations are known to those of skill in the art.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, an “oily” liquid is one which comprises a carbon-containing liquid molecule and which exhibits a less polar character than water.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The terms oral rinse and mouthwash are used interchangeably herein.

A pharmaceutical composition of the invention may be prepared, packaged, or sold in a formulation suitable for oral or buccal administration. Such a formulation may comprise, but is not limited to, a gel, a liquid, a suspension, a paste, toothpaste, a mouthwash or oral rinse, and a coating. For example, an oral rinse of the invention may comprise a compound of the invention at about 1.4%, chlorhexidine gluconate (0.12%), ethanol (11.2%), sodium saccharin (0.15%), FD&C Blue No. 1 (0.001%), peppermint oil (0.5%), glycerine (10.0%), Tween 60 (0.3%), and water to 100%. In another embodiment, a toothpaste of the invention may comprise a compound of the invention at about 5.5%, sorbitol, 70% in water (25.0%), sodium saccharin (0.15%), sodium lauryl sulfate (1.75%), carbopol 934, 6% dispersion in (15%), oil of spearmint (1.0%), sodium hydroxide, 50% in water (0.76%), dibasic calcium phosphate dihydrate (45%), and water to 100%. The examples of formulations described herein are not exhaustive and it is understood that the invention includes additional modifications of these and other formulations not described herein, but which are known to those of skill in the art.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Methods of Assaying Combination Compositions

An established method for evaluating the effects of drugs that act on breathing control is to create closed systems where the key factors that affect breathing can be tightly controlled and monitored. For example, control systems are established for oxygen concentration, carbon dioxide concentration and atmospheric pressure.

For animal-based evaluations, systems are available that allow for whole body or nose only evaluation of multiple respiratory function measurements. There are also established animal models (e.g., guinea pig, dog, rodent) of respiration in combination with allergy, inflammation, COPD and narcotic analgesic use. By way of a non-limiting example, Lovelace Respiratory Research Institute (Albuquerque, N. Mex.) has extensive experience in establishing such models as part of evaluation for new drugs and environmental exposure purposes.

Similar systems have been established for human testing. Hildebrandt et al (Blood 2002; 99:1552-1555) described a protocol that was used for evaluation of N-acetylcysteine under varying conditions of oxygen and carbon dioxide concentrations. In addition, the United States military (Naval Aerospace Medical Research Command, Pensacola Fla., US Army Research Institute of Environmental Medicine, Natick, Mass.) has developed methods that include both whole body and face only exposure/monitoring systems (Sausen et al. Aviat Space Environ Med 2003; 74: 1190-7).

Finally, hospitalized patients who are connected to mechanical ventilation devices represent an opportunity to closely evaluate the effects of drugs on respiration. Levels of oxygen and carbon dioxide can be controlled in an environment where respiration parameters are measured on a minute-by-minute basis.

In addition to the animal and human-based systems described above there is an emerging field where certain biochemical markers are used to indicate chronic oxidative stress resulting from hypoxia. An example is the use of various isoprostanes to indicate oxidative stress (Cracowski J L and Durand T. Fundam Clin Pharmacol 2006; 20:417-27).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A therapeutic composition for stabilizing breathing rhythm, said therapeutic composition comprising:
a. A first composition comprising a S-nitrosothiol first compound; and
b. A second composition comprising a second compound that is not an S-nitrosothiol compound, wherein said second compound has the activity of stabilizing breathing rhythm.

2. A method of stabilizing the breathing rhythm of a mammal, said method comprising administering to a mammal the therapeutic composition of claim 1.

3. The therapeutic composition of claim 1, wherein said second compound is a compound which has the ability to stabilize breathing rhythm.

4. The method of claim 3, wherein said second compound is selected from the group consisting of a carbonic anhydrase inhibitor, a respiratory stimulant, a narcotic antagonist, and a hormone.

5. The therapeutic composition of claim 1, wherein said second compound is a compound which has the activity of increasing the patency of the upper airway.

6. The method of claim 5, wherein said second compound is selected from the group consisting of a serotonin agonist, a serotonin antagonist, a tetracyclic antidepressant, a agent which acts on dopamine, and an agent which acts on norepinephrine.

7. The therapeutic composition of claim 1, wherein said second compound is a compound which has the activity of promoting wakefulness.

8. The therapeutic composition of claim 1, wherein said second compound is a compound which has the activity of decreasing seizures.

9. The therapeutic composition of claim 1, wherein said second compound is a compound which has the activity of increasing the patency of the upper airway by decreasing inflammation.

10. The method of claim 9, wherein said second compound is selected from the group consisting of an antihistamine, a leukotriene antagonist, a 5-lipoxygenase inhibitor, a steroid, and a COX-2 inhibitor.

11. The therapeutic composition of claim 1, wherein said second compound is a compound which has the activity of decreasing respiratory drive.

12. The method of claim 11, wherein said second compound is selected from the group consisting of an opioid analgesic, a sedative hypnotic, and a general anesthetic.

13. The therapeutic composition of claim 1, wherein said second compound is a compound which has the activity of improving lung function.

14. The method of claim 13, wherein said second compound is selected from the group consisting of a steroid, a bronchodilator, and an anticholinergic.

15. The composition of claim 1, further comprising a third compound, wherein said third compound is an S-nitrosothiol compound.

16. The composition of claim 1, further comprising a third compound, wherein said third compound is not an S-nitrosothiol compound.

17. A method stabilizing the breathing rhythm of a mammal, said method comprising administering to a mammal the therapeutic composition of claim 8.

18. A method of stabilizing the breathing rhythm of a mammal, said method comprising administering to a mammal the therapeutic composition of claim 1, said method further comprising treating said mammal with a ventilation assist device.

19. The method of claim 18, wherein said ventilation assist device is selected from the group consisting of a mechanical ventilator, a CPAP device and a BiPAP device.

20. A pharmaceutical composition comprising the therapeutic composition of claim 1 and a pharmaceutically-acceptable carrier.

21. A method of stabilizing the breathing rhythm of a mammal, said method comprising administering to a mammal the therapeutic composition of claim 13.

22. The method of claim 21, wherein said route of administration is selected from the group consisting of parenteral, oral and buccal.

23. The method of claim 22, wherein said parenteral route of administration is selected from the group consisting of transdermal, intravenous, intramuscular, and intradermal.

24. The method of claim 22, wherein said composition is administered by at least two routes of administration.

25. A method of increasing minute ventilation (V_{E}) at the level of the brainstem respiratory control centers in the nucleus tractus solitarius of an individual, said method comprising the step of administering to said individual a therapeutic composition comprising:
a. A first composition comprising a S-nitrosothiol first compound; and
b. A second composition comprising a second compound that is not an S-nitrosothiol compound, wherein said second compound has the activity of increasing minute ventilation (V_{E}) at the level of the brainstem respiratory control centers in the nucleus tractus solitarius.

26. A method stabilizing the breathing rhythm of a mammal, said method comprising administering to a mammal the therapeutic composition of claim 9.
